(19) United States
(12) Patent Application Publication
(10) Pub. No.: US 2007/0276210 A1
(43) Pub. Date: Nov. 29, 2007

(54) Apparatus and method for measuring myocardial oxygen consumption

(76) Inventor: Guillermo Gutierrez, McLean, VA (US)

Correspondence Address: Holland & Knight LLP, Suite 100, 2099 Pennsylvania Ave., Washington, DC, 20707, US

(21) Appl. No.: 11/707,138

(22) Filed: Feb. 16, 2007

Related U.S. Application Data
(63) Continuation-in-part of application No. 10/987,505, filed on Nov. 12, 2004, now Pat. No. 7,181,260.
(60) Provisional application No. 60/520,280, filed on Nov. 14, 2003.

(51) Int. Cl. A61B 5/00

(52) U.S. Cl. 600/325

(57) ABSTRACT

The present invention relates to a method to determine a cardiac characteristic determined at a proximal end and a distal end of a pulmonary artery catheter. The present invention also relates to a method of treating a patient based on a determination of a determined difference between a cardiac characteristic a proximal end and a distal end of a pulmonary artery catheter.

### RELATED APPLICATION DATA

This invention claims priority from provisional application Ser. No. 60/520,280 filed on Nov. 14, 2003 entitled Novel Method to Measure Myocardial Oxygen Consumption in Critically 111 Patients, the contents of which are incorporated herein by reference, and from U.S. Ser. No. 10/987,505 that has a filing date of Nov. 12, 2004.

### BACKGROUND OF THE INVENTION

1. Field of the Invention

This invention relates to an apparatus and method for measuring myocardial oxygen consumption. More particularly, the present invention relates to determining myocardial oxygen consumption by comparing oxygen saturation in atrial (or central veins) and mixed venous blood.

2. Description of the Background Art

Pulmonary artery catheters (“PACs”) are widely used for patient diagnosis and for hemodynamic and therapeutic monitoring. One of the most widely used PACs is the Swan-Ganz catheter. The Swan-Ganz catheter, a version of which is disclosed in U.S. Pat. No. 3,995,623 to Blake, includes a flexible tube (enclosing multiple lumina) that is designed to be flow-directed through a patient's heart by a distal balloon. The catheter is adapted to be delivered through the right atrium and right ventricle with the distal end positioned within the pulmonary artery.

The Swan-Ganz catheter includes first and second lumina for use in measuring blood pressures in the pulmonary artery and right atrium respectively. A third lumen is used for inflating the balloon at the distal end of the catheter. A fourth lumen is included for housing a thermistor that is used in monitoring blood temperature and in determining cardiac output. The fourth lumen also houses the wires associated with electrodes that are included for monitoring intraatrial and intraventricular electrograms. The Swan-Ganz catheter has been a useful tool in diagnosing complex cardiac arrhythmias.

A more recent PAC construction is disclosed in U.S. Pat. No. 6,532,378 to Saksena. In one embodiment, the PAC of Saksena includes a series of defibrillation electrodes interspersed with mapping electrode pairs at the distal end of the catheter. Proximal to the defibrillation and mapping electrodes are a series of sense electrodes and additional defibrillation electrodes. The catheter is used for indirect left atrial mapping from the left pulmonary artery and is also used in defibrillating or cardioverting the heart.

Each of the above referenced inventions is useful in providing a physician with information on the mechanical functioning of a patient's heart. However, none of the aforementioned PACs can be used to measure the rate of oxygen consumption by the heart, or myocardial VO2, whereby a physician may gain an understanding of the energy costs associated with the heart's performance. Measuring myocardial VO2 is significant because a decrease in myocardial VO2 may have serious consequences for critically ill patients. Heretofore, there has been no practical way to obtain myocardial VO2 measurements in critically ill patients.

### SUMMARY OF THE INVENTION

It is an objective of the present invention to provide a method of determining a cardiac characteristic, such as, for example, the state of oxygenation of the heart, comprising: measuring a metabolite indicator at a distal end of a pulmonary artery catheter; measuring the metabolite indicator at a proximal end of the pulmonary artery catheter; and determining a cardiac characteristic based on the measurements of the metabolite indicator.

It is also an object of the present invention to provide a method of treating a patient comprising: measuring a metabolite indicator at a distal end of a pulmonary artery catheter; measuring the metabolite indicator at a proximal end of the pulmonary artery catheter; determining a cardiac characteristic based on the measurements of the metabolite indicator; and treating a patient based on the determination.

### BRIEF DESCRIPTION OF THE DRAWINGS

FIG. 1 is a partial cross sectional view of a catheter usable in embodiments of the present invention.

FIG. 2 is a view of the catheter usable in embodiments of the present invention.

FIG. 3 is a view of a computer usable in embodiments of the present invention.

FIG. 4 is a block diagram illustrating an optical sensor usable in embodiments of the present invention.

FIG. 5 is a block diagram illustrating blood flow and oxygen content from the coronary artery and right atrium into the pulmonary artery.

FIG. 6 is a graph illustrating the relationship between myocardial consumption and differential oxygen saturation between atrial or central venous and mixed venous blood.

FIG. 7 illustrates a first-order mass transport model of the circulation.

FIG. 8 shows ΔSO2 plotted as a function of ERraO2. Also shown is the linear regression of the data (ΔSO2%=16.4-43.6 ERraO2; R=0.55; p<0.001). The intersection of this line with the abscissa defines the population mean myocardial O2 extraction ratio EmO2, in this case 0.38.

FIG. 9 shows a calculated MVO2 plotted as a function of coronary perfusion pressure, cardiac output, left ventricular stroke work index (LVSWI) and the rate-pressure product.

FIG. 10 shows sequential measures of MVO2 (solid line) and the rate pressure product (RPP×10-3; dashed line) plotted individually for study participants in whom three or more samples were obtained during the course of the study. Individual R values range from 0.54 to 0.97 with a weighted average correlation ρ=0.73.

FIG. 11 shows a second-order model of the circulation. The definitions of the various model parameters are shown in Table 1, and F is split into a flow fraction directed to the heart (H) and a flow fraction directed to a virtual compartment (K). The virtual compartment accounts for the effect of incomplete mixing of IVC and SVC blood on the PAC proximal port blood sample.

Similar reference characters refer to similar parts throughout the several views of the drawings.

### DETAILED DESCRIPTION OF THE PREFERRED EMBODIMENTS

The present invention relates to a pulmonary artery catheter (“PAG”) and its use in determining a cardiac characteristic, such as, for example, myocardial oxygen consumption. Myocardial oxygen consumption is of critical importance because decreased myocardial energy utilization during acute illness may lead to tissue hypoperfusion, multiple organ failure, and eventually death. The inventor has discovered that myocardial oxygen consumption is a function of the difference in oxygen levels in atrial (or central veins) and mixed venous blood. The inventor has further discovered that differences in lactate, glucose or any other measurable blood concentration metabolite in atrial or central venous blood and mixed venous blood can also be used in determining myocardial energy metabolism.

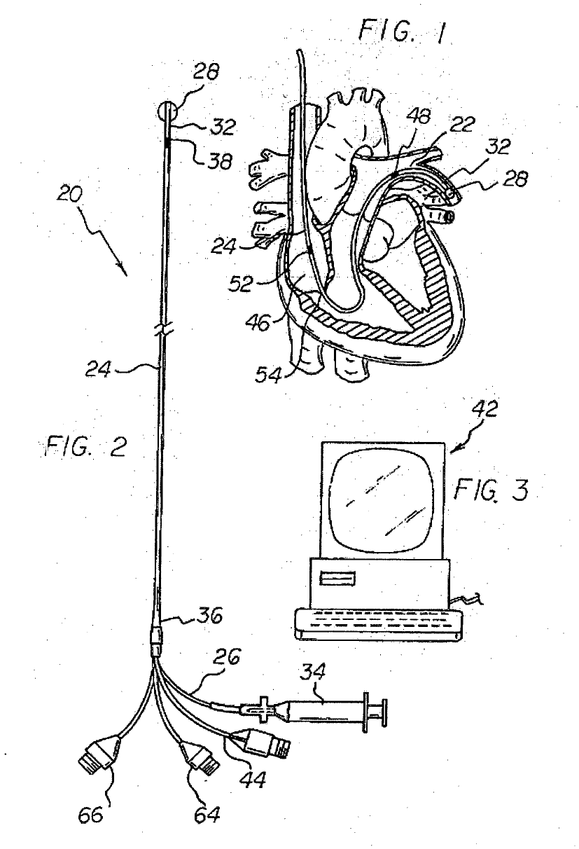

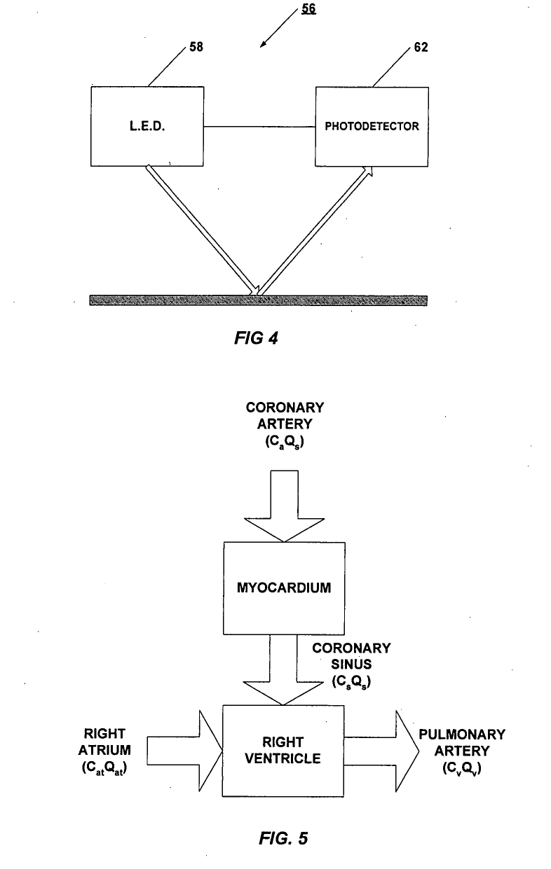

The measurements necessary to calculate myocardial oxygen consumption are carried out by way of a PAC. FIG. 1 illustrates PAC 20 positioned within the pulmonary artery 22 of a patient's heart. As is conventional, PAC 20 of the present invention is constructed from an elongated flexible tube 24. Tube 24, which may be coated with a material to facilitate its insertion into a patient's vein, houses a series of lumina each of which serves a different diagnostic or therapeutic purpose.

For example, one lumen 26 is used to selectively inflate or deflate a balloon 28 at the distal end 32 of PAC 20. Balloon 28 is preferably formed from a flexible material that expands upon receiving a fluid through lumen 26. This fluid can be selectively injected into or withdrawn from balloon 28 via a syringe 34 at the proximal end 36 of PAC 20. When inflated, balloon 28 allows PAC 20 to be “flow-directed” to a patient's heart.

PAC 20 additionally includes a thermistor 38 positioned adjacent to balloon 28. The use of thermistors in PACs is known in the art and is generally described in U.S. Pat. No. 3,995,623 to Blake. Electricat leads (not shown) are used to couple thermistor 38 to a microprocessor 42, or other diagnostic equipment, as will be described in greater detail hereinafter. An additional lumen 44 is included to shield the leads of the thermistor 38. Thermistor 38 is used to monitor blood temperature and also allows total cardiac output to be determined by way of thermodilution. As will be elaborated upon hereinafter, total cardiac output is one factor used in calculating myocardial oxygen consumption.

One of the other factors needed to determine myocardial oxygen consumption is the difference in oxygen content between the right atrium 46 and the pulmonary artery 22 (i.e. between atrial (or central veins) and mixed venous blood). This difference is measured via two oxygen sensors positioned along the length of PAC 20. Namely, a first oxygen sensor 48 is located at a distal end 32 of PAC 20, while second sensor 52 is located proximal to the first. Locating second sensor 52 approximately 30 centimeters or more from distal end 32 of PAC 20 is preferred. With PAC 20 properly positioned within a patient's heart, first sensor 48 is positioned for readings within pulmonary artery 22 and second sensor 52 is positioned for readings within right atrium 46. Ideally, second sensor 52 will be located about 3-4 centimeters above the tricuspid valve 54. Nonetheless, oxygen saturation can also be measured from the superior vena cava, upstream from the atrium. This is because measuring oxygen saturation in a superior vena cava (central venous blood) provides the same information as measuring atrial blood.

The sensors can measure blood oxygen content in any number of ways. For example, chemical sensors can be employed in making the measurements. Additionally, the sensors need not be located on the length of PAC 20, rather direct blood sampling may be used in making the necessary measurements. In the preferred embodiment, however, optical sensors 56 are employed. One suitable optical sensor is described in U.S. Pat. No. 4,684,245 to Goldring. The sensor described in the '245 patent includes a series of light emitting diodes (“LEDs”) and a photodetector.

LEDs 58 are used to radiate infrared light into an adjacent blood sinus whereby the sensor's photodetector 62 can detect infrared absorption by the blood. The oxygen level in the blood can be determined from the level of infrared absorption. The optical sensors 56 are operatively coupled to microprocessor 42 for use in storing and processing the detected oxygen levels. The catheter includes additional lumina (64, 66) for housing the leads to optical sensors 48 and 52.

The present invention can employ any microprocessor 42 suitable for carrying out the algorithms described herein. The microprocessor can either be carried on-board PAC 20 or it can be a physically separate stand alone computer, such as a laptop. Microprocessor 42 is used in computing the oxygen consumption by the heart or myocardial VO2 on the basis of the following equation:
V0₂=Qv(Cat−Cv)+QvFₛ(Ca−Cat) Eq. 1

where,

Qᵥ=pulmonary artery blood flow or total cardiac output; this value is obtained from thermistor 38.

Cat=Oxygen (Oz) content in atrial (or central veins) blood; this value is obtained from second oxygen sensor 52 located within the right atrium 46.

Cv=Oxygen (62) content in the pulmonary artery blood; this value is obtained from first oxygen sensor 48 located within pulmonary artery 22.

Cₐ=Oxygen (02) content in the coronary artery (arterial saturation); this value is obtained from a pulse oximeter, through a noninvasive and standard technique known to most Intensive Care Units and anyone skilled in the art;

Fₛ=fraction of total blood flow going to the myocardium; this value is unknown for any particular patient, but it can safely be approximated to be between 0.05 and 10.

Equation 1 is derived from the principle of conservation of mass known as Fick's Principle as noted in the mass transport model depicted in FIG. 5 and the following equation:
VO₂=CaQs−C_{S}Q_{S} Eq. 2

The various O2 contents referenced in Equation 1 are based on the well known composition of O2 within blood. That is, total O2 is comprised of a percentage of O2 bound to hemoglobin and a percentage of O2 dissolved in plasma. The following equation reflects the known composition of O2 within blood:
O2 Content=1.34×hemoglobin concentration×O2 saturation+0.0003 PO2

As can be observed from Equation 3, the percentage of O2 from dissolved oxygen is quite small and can be neglected. In doing so, Equation 1 can be simplified as follows:
(V0₂)=KQvKSat−Sv)+Fs(Sa−Sat)] Eq. 4

Here,
K=1.34×hemoglobin concentration.
Sat=Oxygen saturation in atrial (or central veins) blood.
Sᵥ=Oxygen saturation in the pulmonary artery blood.
Sₐ=Arterial Oxygen saturation.
   A close approximation to VO2 is obtained by:
   VO₂=KQᵥ(Sₐₜ−Sv) Eq. 5
   Microprocessor 42 can employ either Equations 1 or 4 in computing myocardial VO₂ consumption.

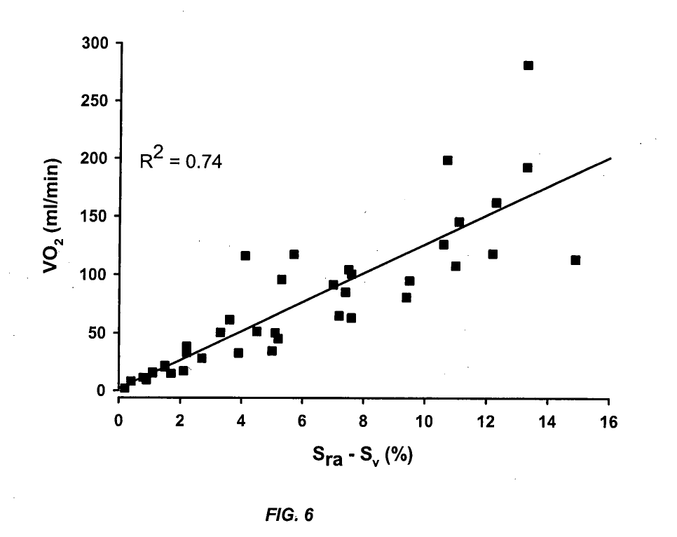

FIG. 6 is a graph illustrating data taken from 50 patients in whom a single determination of VO₂ was made. FIG. 6 is a graph showing the same data but shows VO₂ plotted as a function of the difference in O₂ saturation between the first and second oxygen sensors. VO₂ was calculated using Equation 4 with Fs=0.05.

FIG. 6 illustrates that VO₂ is proportional to the difference in the saturation between the proximal and distal oxygen sensors. Therefore, the difference in O2 saturation between atrial (or central veins) and mixed venous blood (Sₐt−Sᵥ) could be used to monitor relative changes in VO2 in a given patient without the need to measure Qv or hemoglobin saturation. It is also possible to monitor changes in VO2 continuously by use of infrared optics placed in the tip and the atrial (or central veins) region of the PAC.

The principles presented herein can also be used to measure atrial (or central veins) and mixed venous blood differences in lactate, glucose and any other measurable blood concentration metabolite to serve as a measure of myocardial metabolism.

This is because the healthy myocardium generates its energy supply from the oxidation of fatty acids with the balance of energy production derived from the oxidation of glucose and lactate. Under aerobic conditions there is net lactate extraction from the coronary circulation with the oxidation of lactate accounting for 10% to 20% of the myocardial energy production, a proportion that increases substantially in septic patients. Given the heart's penchant for lactate as a metabolic substrate, coronary venous blood lactate concentration usually is lower than central venous blood lactate concentrate. The mixing of these effluents in the right ventricle should result in a declining blood lactate concentrate gradient from right atrium to pulmonary artery.

The metabolic response of the heart to acute illness often determines patient survival. Uncompensated cardiac failure can lead to systemic hypoperfusion, tissue hypoxia, multiple system organ failure and death. To assure tissue perfusion, current treatment of sepsis and other shock states calls for the infusion of fluids, vasopressors and inotropic agents. These interventions, while increasing cardiac output and systemic oxygen delivery (DO₂), are also likely to increase cardiac work and perhaps affect adversely myocardial aerobic metabolism

Mechanical parameters of myocardial performance, such as contractility and cardiac output, are measured routinely in critical ill patients using echocardiography or, more invasively, with a PAC. On the other hand, there is no practical technique available with which to monitor myocardial energy metabolism in the ICU. Given the heart's predilection for oxidative phosphorylation as the main source of ATP, myocardial energy generation may be inferred from its rate of O₂ uptake (MVO₂) Therefore, a method capable of measuring MVO₂ in critically ill patients could provide the means with which to monitor the effect of therapeutic interventions on cardiac energy metabolism. This monitoring modality would be particularly useful when treating patients in septic or cardiogenic shock, conditions usually associated with impaired myocardial function

Presently, measurement of MVO₂ in the clinical setting is an onerous task, one that requires knowledge of coronary sinus blood O₂ saturation (S_{cs}O₂), as well as of total coronary blood flow. S_{cs}O₂ may be measured from blood samples drawn from a catheter placed in the coronary sinus, a demanding procedure in the best of hands. Moreover, accurate measures of coronary sinus blood flow are exceedingly difficult to obtain More accurate techniques, such as magnetic resonance imaging⁷ and contrast echocardiography have been used to estimate total coronary blood flow, but these techniques are not suitable for ICU monitoring.

Others have reported the existence of an O₂ saturation gradient (ΔSO₂) from right atrium to pulmonary artery in critically ill patients. The magnitude of ΔSO₂ is approximately 5%, although wide variations are found among individuals or even in the same person when measurements are taken at different times The mechanism resulting in ΔSO₂ is not known, but it is probable that mixing of right atrial (or central veins) with coronary venous blood plays an important role in its development. On this basis, it is reasonable to hypothesize that ΔSO₂ represents a physiological signal that bears some degree of relationship to coronary venous SO₂ and therefore, to MVO₂.

Should the above hypothesis hold true, then it may be possible to use the PAC to monitor MV₂ in critically ill individuals by computing ΔSO₂ from blood samples drawn from the catheter's proximal and distal ports. The purpose of this study was to explore this possibility by developing a mathematical expression relating ΔSO₂ to MVO₂, and comparing the results of this expression to parameters of myocardial energy utilization in a heterogeneous group of critically ill individuals.

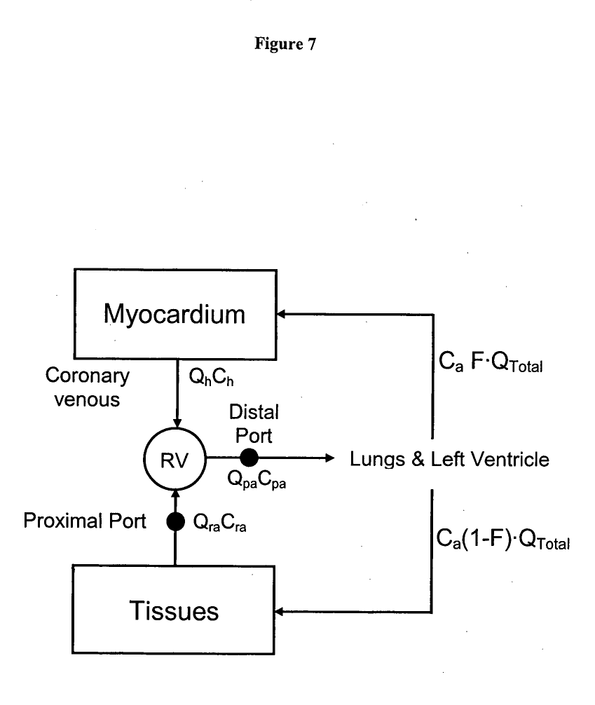

The derivation of the equations used to calculate MVO₂ is shown below. These equations are based on the first-order mass transport model of FIG. 7, where ‘Q’ represents blood flow and ‘C’ the O₂ content of blood. The subscripts ‘a’, ‘ra’, ‘pa’ and ‘h’ refer to arterial, right atrial (or central veins), pulmonary artery and coronary venous blood, respectively.

The equation used to calculate MVO₂ is derived using the first-order mass transport model shown in FIG. 7, where ‘Q’ represents blood flow and ‘C’ the O₂ content of blood. Referring to FIG. 7, the definitions of the various model parameters are shown in Table 1. The model assumes perfect mixing of blood from the superior and inferior vena cava prior to reaching the Proximal Port sampling site, with further mixing with coronary venous blood occurring in the right ventricle. O2 consumption takes place in the “Tissues” and “Myocardium” compartments. Sampling of mixed venous blood occurs in the Distal Port located in the pulmonary artery. From conservation of flow and conservation of mass principles,
Qₚₐ=Qᵣₐ+Qₕ (6a)
and
QᵣₐCᵣₐ+QₕCₕ=QₚₐCₚₐ (7a)
Combining the above equations yields,
QᵣₐCᵣₐ+QₕCₕ=(Qᵣₐ+Qₕ)Cₚₐ (8a)
According to Fick's Principle, myocardial O₂ consumption is,
MVO₂=Qₕ(Cₐ−Cₕ) (9a)
Solving (4a) for Cₕ, and substituting into (3a),
Qᵣₐ(Cᵣₐ−Cₚₐ)=Qₕ[Cₚₐ−Cₐ+MVO₂/Qₕ] (10a)
Taking Qₚₐ as the total cardiac output (Qₜₒₜₐₗ) and defining F as the fraction of Qₜₒₜₐₗ directed to the heart, F=Qₕ/Qₜₒₜₐₗ, yields the following expression for MVO₂,
MVO₂=Qₜₒₜₐₗ[Cᵣₐ−Cₚₐ+F(Cₐ−Cᵣₐ)]ml·min⁻¹ (11a)
Dividing the above expression by the myocardial O₂ delivery (CₐQₕ=FCₐQₜₒₜₐₗ) yields the myocardial O₂ extraction ratio,
ERₘO₂=[(Cᵣₐ−Cₚₐ)/F+Cₐ−Cᵣₐ]/Cₐ (12a)
Neglecting the effect of plasma dissolved O₂ on the calculation of blood O₂ contents, and noting that ΔSO₂=SᵣₐO₂−SₚₐO₂, yields expressions for MVO₂ and of ERₘO₂ in terms of ΔSO₂ and the measured O₂ saturations,
MVO₂=13.9·[Hb]·Qₚₐ[ΔSO₂+F(SₐO₂−SᵣₐO₂)]ml·min⁻¹ (13a)
ERₘO₂=[ΔSO₂/F+SₐO₂−SᵣₐO₂]/SₐO₂ (14a)
where [Hb] is the blood hemoglobin concentration.
According to the model, MVO₂ is calculated as,
MVO₂=13.9·[Hb]·Qₚₐ[ΔSO₂+F(SₐO₂−SᵣₐO₂)]ml·min⁻¹ (15)
Where F is the fraction of the cardiac output directed to the myocardium; ΔSO₂=SᵣₐO₂−SₚₐO₂; and [Hb] is the blood hemoglobin concentration. The myocardial O₂ extraction ratio is,
ERₘO₂=[ΔSO₂/F+SₐO₂−SᵣₐO₂]/SₐO₂ (16)
Equation (16) contains two undefined unknowns, ERₘO₂ and F. These parameters may be estimated by solving equation (16) for ΔSO₂,
ΔSO₂=F·SₐO₂·(ERₘO₂−ERᵣₐO₂) (17)
where ERᵣₐO₂=1−SᵣₐO₂/SₐO₂. Equation (17) traces a line with slope F·SₐO₂ whose intercept (at ΔSO₂=0) equals ERₘO₂.

The above was validated using a prospective, sequential study conducted at The George Washington University Hospital intensive care unit. The Institutional Review Board approved of the study and informed consent was obtained from the patient or from the next of kin. Critically ill individuals older than 18 years of age of either sex in whom a PAC was required to guide fluid therapy were enrolled in the study. Excluded were patients with uncorrected valvular incompetence or intra-cardiac shunts. Physicians not involved in the study determined the clinical need for using PACs in these patients. PACs were inserted using standard technique through the internal jugular or femoral approach. All patients had an arterial line inserted as part of their ICU management. After discarding the first 2 ml of blood, one ml aliquots were drawn in random order and in rapid succession from the arterial line and the proximal and distal ports of the PAC. The latter samples were drawn with the catheter balloon deflated. This was followed by measurement of heart rate, arterial, pulmonary artery, central venous, and PA occlusion pressures (PAOP) and cardiac output in triplicate by the thermodilution method. Measurements were obtained within 24 hours of catheter insertion and daily thereafter, until the PAC was removed. Blood samples were immediately analyzed in triplicate for oxyhemoglobin concentration ([Hb]) and O₂ saturation (IL682 CO-Oximeter, Instrumentation Laboratories, Lexington, Mass.).

In analyzing the obtained data, Cardiac index (CI), myocardial perfusion pressure (PP), rate-pressure product (RPP), left ventricular stroke work index (LVSWI), systemic O₂ delivery (DO₂), systemic O₂ consumption (VO₂), and the systemic O₂ extraction ratio (ERO₂) were computed according to standard formulae. Paired Student's t-test was used to compare SᵣₐO₂ to SₚₐO₂. The relationship of MVO₂ to the various computed and measured parameters was analyzed by Spearman's correlation analysis. Data are shown as mean±SD with p<0.05 taken as a significant difference.

Sixteen critically patients with various medical and post-surgical conditions were enrolled in the study. Descriptive demographic data for the group and number of samples taken per patient are shown in Table 2. Table 2, presents the descriptive demographics and number of blood samples sets obtained in the patients enrolled in the study (n=16); and CABG=Coronary artery bypass grafting.

TABLE 2

| No. | Sex | Age | Diagnosis | APACHE II | Samples |
|---|---|---|---|---|---|
| 1 | F | 69 | Acute respiratory failure | 12 | 1 |
| 2 | M | 75 | Pneumonia | 9 | 1 |
| 3 | M | 55 | Acute myocardial infarction | 10 | 2 |
| 4 | M | 80 | Post CABG | 18 | 1 |
| 5 | M | 58 | Heart failure | 7 | 2 |
| 6 | F | 45 | Post CABG | 2 | 1 |
| 7 | M | 48 | Sepsis, multiple myeloma | 23 | 5 |
| 8 | F | 56 | Sepsis, pneumonia, breast | 23 | 6 |
| | | | cancer | | |
| 9 | F | 72 | Post CABG | 11 | 2 |
| 10 | M | 76 | Post CABG | 10 | 2 |
| 11 | M | 49 | Pneumonia | 9 | 4 |
| 12 | F | 76 | Aortic valve repair | 8 | 3 |
| 13 | M | 85 | Pulmonary hypertension | 15 | 3 |
| 14 | M | 72 | Heart failure | 10 | 2 |
| 15 | M | 75 | Pulmonary hypertension | 5 | 1 |
| 16 | M | 68 | Heart failure | 15 | 5 |

The mean age was 66±12 years and the APACHE II score 16±6. Eleven were men. The number of samples per patient varied according to the time the PAC remained in place, ranging from one to six, yielding 41 sets of simultaneous arterial, RA and PA samples.

Table 3 shows the combined hemodynamic and O₂ transport data for all study participants. Table 3 presents Hemodynamic and systemic O₂ transport parameters; and PAOP=Pulmonary artery occlusion pressure; SVRI=Systemic vascular resistance index; PVRI=Pulmonary vascular resistance index; PRP=Double product; LVSWI=Left ventricular stroke work index; DO₂=Systemic O₂ delivery; VO₂=Systemic O₂ consumption; ERO₂=Systemic O₂ extraction ratio.

TABLE 3

| | Mean ± SD | Median | Min | Max |
|---|---|---|---|---|
| Body Temperature (° C.) | 36.6 ± 0.7 | 36.5 | 35.0 | 38.1 |
| Heart rate (bpm) | 98 ± 15 | 96 | 67 | 130 |
| Cardiac Output (L · min⁻¹) | 5.8 ± 1.8 | 5.4 | 2.9 | 12.8 |
| Cardiac Index | 3.1 ± 0.9 | 3.1 | 1.5 | 6.8 |
| (L · min⁻¹ · m⁻²) | | | | |
| Mean Arterial Pressure | 84 ± 12 | 83.0 | 63.0 | 110 |
| (mmHg) | | | | |
| Mean Pulmonary Pressure | 34 ± 11 | 33 | 12 | 56 |
| (mmHg) | | | | |
| PAOP (mmHg) | 20 ± 8 | 18 | 6 | 45 |
| Central Venous Pressure | 19 ± 10 | 19 | 1 | 37 |
| (mmHg) | | | | |
| SVRI (dynes · sec⁻¹ · cm⁻⁵) | 1773 ± 605 | 1648 | 874 | 3378 |
| PVRI (dynes · sec⁻¹ · cm⁻⁵) | 385 ± 250 | 313 | 110 | 998 |
| Stroke Volume (mL) | 60 ± 18 | 58 | 30 | 113 |
| Rate Pressure Product × 10⁻³ | 118 ± 24 | 114 | 72 | 170 |
| (mmHg · beats · min⁻¹) | | | | |
| LVSWI (g · m · m⁻²) | 28 ± 11 | 28 | 11 | 64 |
| Hemoglobin (g · dL⁻¹) | 10.1 ± 1.6 | 10.0 | 7.3 | 14.1 |
| VO₂ (ml · min⁻¹) | 238 ± 65 | 211 | 129 | 390 |
| DO₂ (ml · min⁻¹) | 761 ± 219 | 718 | 360 | 1,294 |
| ERₚₐO₂ | 0.32 ± 0.08 | 0.32 | 0.18 | 0.53 |

Referring to Table 3, there was a wide variation in all parameters, reflecting the heterogeneity of clinical diagnoses in the patient population. Table 4 shows arterial, RA and PA blood O₂ saturations and ΔSO₂. SᵣₐO₂ was greater than SₚₐO₂(p<0.01) with ΔSO₂=4.3±6.8%. There was a wide dispersion of individual ΔSO₂ values, ranging from −8.2% to 16.8%.

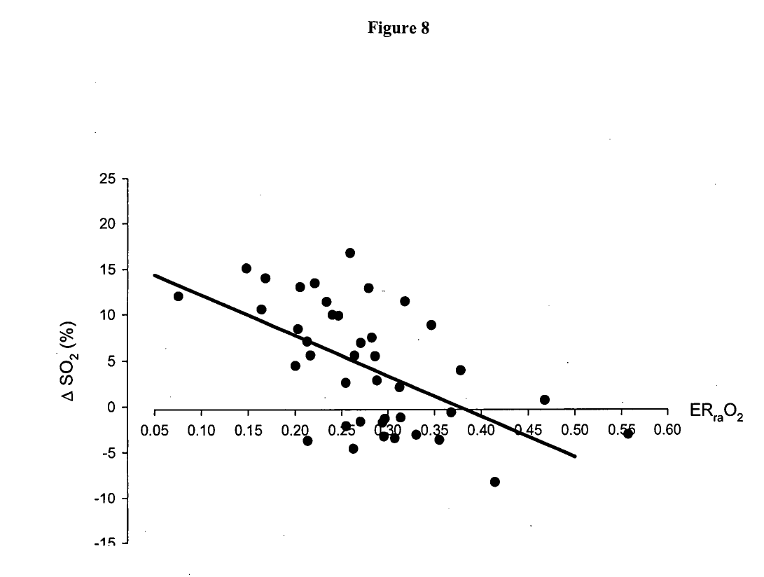

FIG. 8 shows ΔSO₂ plotted as a function of ERᵣₐO₂ (computed as 1−SᵣₐO₂/SₐO₂). Also shown is the linear regression of the data (ΔSO₂%=16.4−43.6 ERᵣₐO₂; R=0.55; p<0.001). The intercepts of this regression line are ΔSO₂=16.4% for ERᵣₐO₂=0 and ERᵣₐO₂=0.38 for ΔSO₂=0. The latter corresponds to the condition where ERₘO₂=ERᵣₐO₂. The myocardial flow fraction F was computed from equation (17) using values for ERᵣₐO₂=0; ΔSO₂=16.4, ERₘO₂=0.38 and SₐO₂=95.6% (the group's mean SₐO₂ from Table 3), resulting in F=0.45. Table 5 lists myocardial O₂ transport parameters calculated using equations (15) and (16) with F=0.45. In Table 5, shows computed myocardial oxygenation parameters assuming F=0.45. The coronary perfusion was calculated as F·Qtotal, and in Table 5, MDO2=Myocardial O2 delivery; MVO2=Myocardial O2 consumption (from equation 10); EmO2=Myocardial O2 extraction ratio (from equation 14).

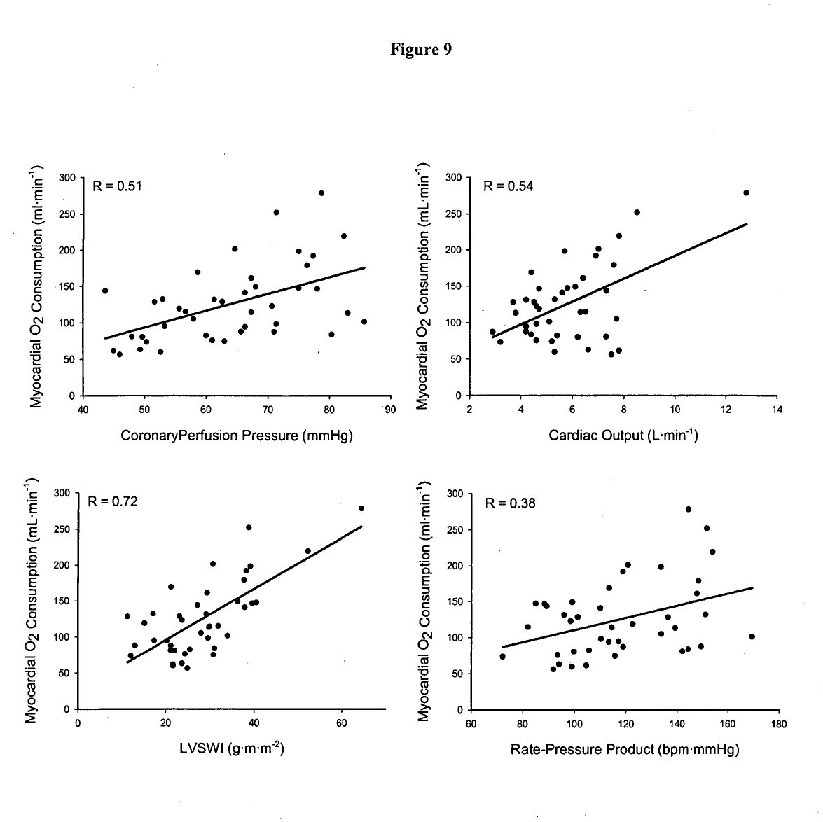

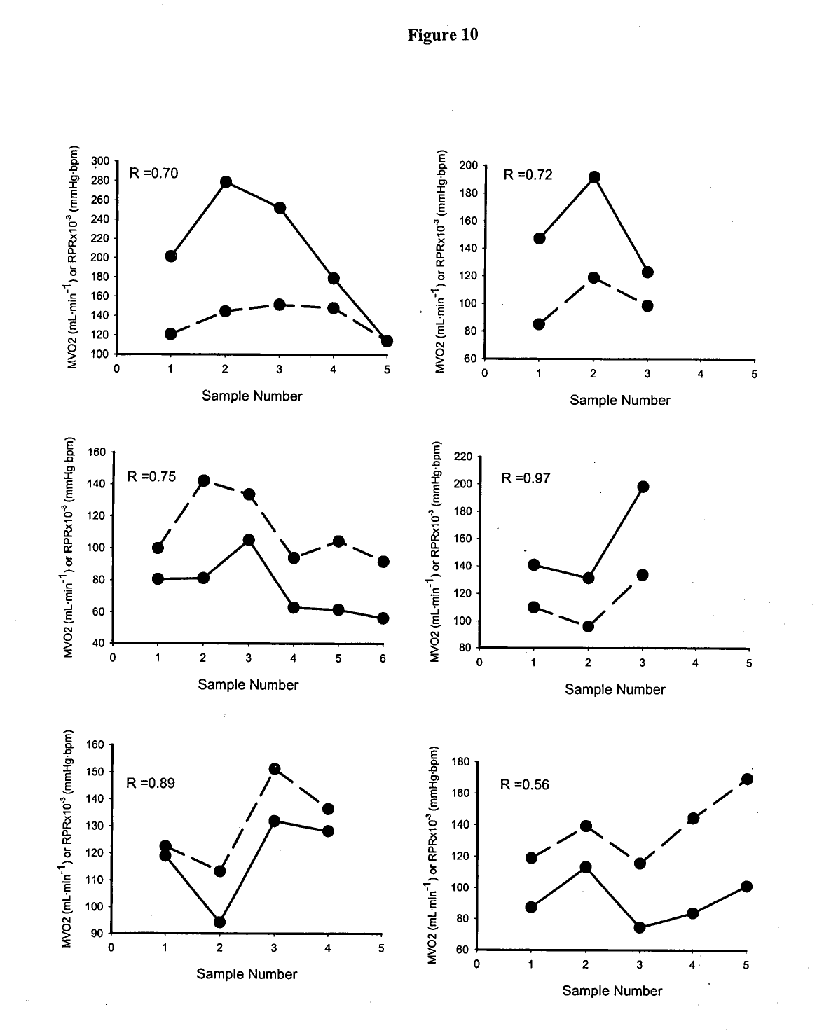

FIG. 9 shows calculated MVO₂, corresponding to each set of blood SO₂ measurements (F=0.45), plotted as a function of PP, CO, LVSWI and RPP, respectively. MVO₂ decreased with decreasing PP (MVO₂=2.3 PP−19.8; R=0.51; p<0.001); and increased in concert with the parameters of cardiac performance CO, LVSWI and RPP. MVO₂ was most closely related to LVSWI (MVO₂=3.6 LVSWI+25.7; R=0.72; p<0.001); with less robust associations noted for CO (MVO₂=15.9 CO+36.0; R=0.54; p<0.01); and RPP (MVO₂=0.8 RPP+26.4; R=0.38; p<0.02). A stronger relationship between MVO₂ and RPP emerges when comparing these variables in individual subjects. This is illustrated in FIG. 10, where sequential measures of RPP and MVO₂ are plotted individually for study participants in whom three or more samples were obtained during the course of the study. Individual R values ranged from 0.54 to 0.97 with a weighted average correlation ρ=0.73. The high individual correlation values shown in FIG. 10 suggests that the dispersion of combined data around the correlation line for RPP and MVO₂ (FIG. 9) reflects mainly the heterogeneous nature of the patient population studied, although other factors also may be involved

The clinical complexity of placing a coronary sinus catheter, as well as the precarious condition of the patients enrolled in the study, precluded the direct measure of MVO₂. Instead, MVO₂ derived from equation (15) was compared to CO, LVSWI and RPP. These hemodynamic parameters are related to the rate of myocardial energy utilization and served as surrogates for direct measures of MVO₂. The significant degree of association found between calculated MVO₂ and these cardiac performance parameters supports the hypothesis that ΔSO₂ mirrors alterations in myocardial energy utilization.

The most striking correlation was that noted between MVO₂ and LVSWI (R=0.72), a marker of cardiac contractility. Although it may be argued that this relationship could have resulted in part from coupling of shared data, since both the calculation of LVSWI and MVO₂ include the cardiac output term, the lesser robustness noted between CO and MVO₂ (R=0.51) belies this argument.

The significant correlation noted between RPP and MVO₂ in individual subjects (ρ=0.73) also supports the hypothesis that ΔSO₂ is related to myocardial energy utilization. RPP is used widely as an index of aerobic myocardial metabolism, based on human and experimental studies that show a strong correlation between RPP and cardiac O₂ uptake. It should be noted that the calculation of RPP (systolic BP×HR) does not incorporate any of the terms used in the calculation of MVO₂, thus eliminating the possibility of data coupling.

Application of the clinical data to the mass transport model of FIG. 15 resulted in a value for F, the fraction of the cardiac output directed to the heart, of 0.45. For a mean population cardiac output of 5.8 L/min (Table 3), this F value predicts a mean coronary flow of 2.6±0.8 L/min, a value that is exceedingly high when compared to published data from experimental and clinical studies. Dhainaut J F, Huyghebaert M F, Monsallier J F, Lefevre G, Dall'Ava-Santucci J, Brunet F, Villemant D, Carli A, Raichvarg D. Coronary hemodynamics and myocardial metabolism of lactate, free fatty acids, glucose, and ketones in patients with septic shock. Circulation 1987;75:533-541, measured coronary sinus flow with a thermodilution technique in septic shock patients and reported it to be approximately 0.2 L/min, corresponding to F=0.03. Cunnion R E, Schaer G L, Parker M M, Natanson C, Parrillo J E. The coronary circulation in human septic shock. Circulation 1986;73:637-644, reported coronary venous flow in individuals with septic shock of 0.45 L/min, with some patients having flows approaching 1.0 L/min. This value, however, results in F=0.10, still far less than that predicted by the model.

A possible explanation for the wide discrepancy noted in calculated and measured F values is that clinical measures of coronary sinus flow underestimate total coronary venous flow. Coronary venous flow is usually assumed to equal coronary sinus flow, the latter obtained by thermodilution tracer techniques, an error-prone method. Moreover, even when measured accurately, coronary sinus flow may underestimate total coronary venous flow. The great cardiac and middle cardiac veins drain into the coronary sinus in only 50% of human hearts and the major epicardial veins drain into the coronary sinus in only 20% of cases. Measures of coronary blood flow obtained simultaneously by phase-contrast magnetic resonance and PET scanning shows that coronary sinus flow accounts for only 65% the total left ventricular venous flow. This difference, however, is not large enough to explain the discrepancy between the model-predicted and clinical estimates of coronary blood flow.

Perhaps a more likely explanation for the high predicted value of F is that the equations of the model described by FIG. 7 oversimplify a highly complex hydrodynamic system, resulting in an overestimate of myocardial blood flow. This first-order model assumes perfect blending of IVC and SVC blood occurring prior to central venous blood reaching the location of the PAC proximal port. Further, mixing of central venous and coronary venous blood occurs exclusively within the right ventricle. The real situation is more complicated. Instead, the anatomical relationship of the inferior vena cava (IVC), the superior vena cava (SVC) and the coronary sinus is such that they empty their venous contents in close proximity in the right atrium. Moreover, this is a dynamic system in which catheter motion may alter the position of the proximal port within the right atrium from beat to beat. In this intricate hydrodynamic system, the position of the proximal port of the PAC, relative to the coronary sinus is of great, and so far not well understood significance.

Another complicating factor in describing the process by which blood mixes in the right atrium is the possibility that (SO₂)_{SVC} may differ from (SO₂)_{IVC}, depending on the patient's clinical condition. Reportedly, (SO₂)_{SVC}≦(SO₂)_{IVC} in individuals not in shock, whereas the opposite occurs in shock states. For example, the condition where (SO₂)_{SVC}>>(SO₂)_{IVC}, coupled with imperfect mixing of the central venous flows, could result in ΔSO₂ being related mainly to the mixing of SVC and IVC blood streams. Under these conditions the first-order model would yield values for MVO₂ and F higher than those warranted by the actual physiological condition.

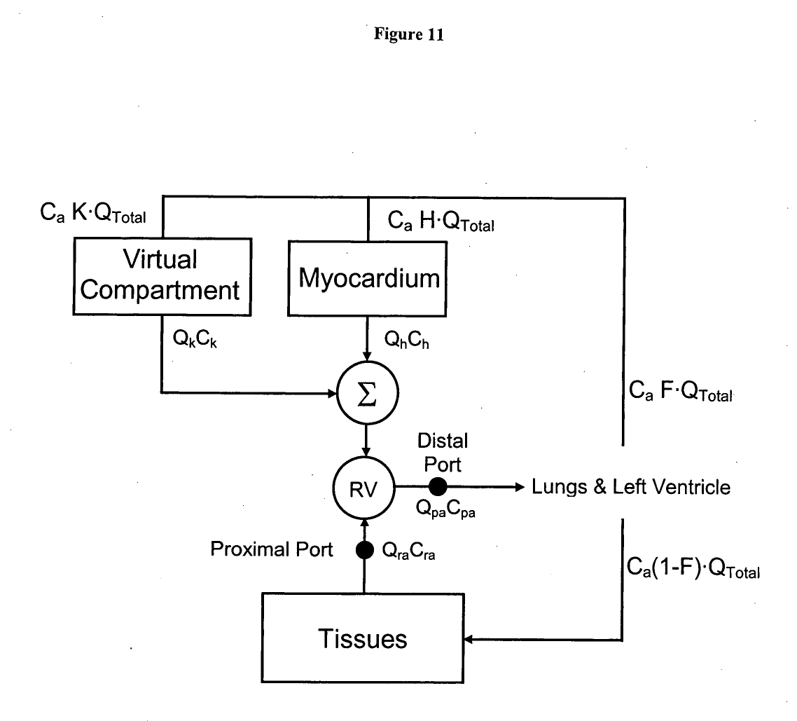

A mathematical model accounting for the above mentioned caveats is a difficult undertaking. Shown in FIG. 11 is a more complex, but less well defined mass transport model that accounts for incomplete mixing and different O₂ saturations for SVC and IVC. According to this model, F is now split into a flow fraction directed to the heart (H) and a flow fraction directed to a virtual compartment (K). The virtual compartment accounts for the effect of incomplete mixing of IVC and SVC blood on the PAC proximal port blood sample. In this model, the value of H may be defined to conform to published reports, as long as the relationship K=F−H is maintained.

For the model configuration shown in FIG. 11 (derivation not shown),
SₕO2=[SₚₐO₂−SᵣₐO₂(1−F)+SₖO₂(H−F)]/H (18)
and
ERₕO₂−1−[SₚₐO₂−SᵣₐO₂(1−F)+SₖO₂(H−F)]/H·SₐO₂) (19)
where Sₖ is the venous saturation of the virtual compartment. As a first approximation, Sₖ may be assigned a value of Sᵣₐ+Δ_{CV}, where Δ_{CV} is the difference between (SO₂)_{SVC} and (SO₂)_{IVC}.

Application of data obtained in the current study to equation (19) is not possible, since it requires several unwarranted assumptions, in particular regarding values for the unknown parameters H and Δ_{CV}. On the other hand, the model of FIG. 11 does provide a theoretical construct to be tested in future studies that include simultaneous drawing of right atrial (or central veins), pulmonary artery, IVC, SVC and coronary sinus blood samples. From a clinical perspective, however, increasing the model's complexity may be of marginal utility in the management of critically ill patients. The first-order model, imperfect as it may be, appears capable of reflecting changes in MVO₂ taking place in these individuals.

A standard PAC can serve to monitor myocardial O₂ uptake. The PAC is a widely used monitoring device and adoption of the simple technique described should is relatively easy. Moreover, it would not be a difficult task to adapt current in vivo oximetry technology to measure ΔSO₂, thus allowing for continuous monitoring of MVO₂ while reducing sources of measuring error. The bioenergetic information provided by ΔSO₂ also may be supplemented by measuring the RA to PA concentration gradients of other parameters involved in myocardial energy metabolism, such as lactate^{25,26}, CO₂, glucose and free fatty acids.

Additional clinical and experimental studies are required to fully understand the physiological principles that relate ΔSO₂ to myocardial O₂ uptake at the heart's rate of energy utilization. These studies should include mapping of central venous, right atrial (or central veins) and coronary sinus blood O₂ saturations under various pathological conditions, as well as the direct measures of MVO₂. This information should better define the relationship of the mass-transport models presented here, and even the relevance of the models themselves, to the determination of MVO₂ in critically ill individuals.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

What is claimed is:

1. A method of determining a cardiac characteristic comprising:
a) measuring a metabolite indicator at a distal end of a pulmonary artery catheter;
b) measuring the metabolite indicator at a proximal end of the pulmonary artery catheter; and
c) determining a cardiac characteristic based on the measurements of the metabolite indicator.

2. A method of determining a cardiac characteristic according to claim 1, wherein the metabolite indicator includes at least one of: SO₂, Lactate, and Glucose.

3. A method of determining a cardiac characteristic according to claim 1, wherein the metabolite indicator includes and indicator that indicates a difference in a metabolite concentration between the distal and proximal ports of the pulmonary artery catheter.

4. A method of treatment comprising:
a) measuring a metabolite indicator at a distal end of a pulmonary artery catheter;
b) measuring the metabolite indicator at a proximal end of the pulmonary artery catheter;
c) determining a cardiac characteristic based on the measurements of the metabolite indicator; and
d) treating a patient based on the determination.

5. A method of treatment according to claim 4, wherein the metabolite indicator includes at least one of: SO₂, Lactate, and Glucose.

6. A method of treatment according to claim 4, wherein the metabolite indicator includes and indicator that indicates a difference in a metabolite concentration between the distal and proximal ports of the pulmonary artery catheter.
